(19) United States
(12) Patent Application Publication
(10) Pub. No.: US 2005/0112764 A1
(43) Pub. Date: May 26, 2005

(54) Sleeping beauty, a transposon vector with a broad host range for the genetic transformation in vertebrates

(76) Inventors: Zoltan Ivics, Berlin (DE); Zsuzsanna Izsvak, Berlin (DE); Hatem Zayed, Berlin (DE)

Correspondence Address: NORRIS, MCLAUGHLIN & MARCUS, P.A., 875 THIRD AVE, 18TH FLOOR, NEW YORK, NY, 10022, US

(21) Appl. No.: 10/258,654

(22) PCT Filed: Apr. 27, 2001
(86) PCT No.: PCT/DE01/01595

(30) Foreign Application Priority Data
Apr. 27, 2000 (DE) 100205534

(51) Int. Cl. A61K 48/00; C12N 15/86; C12N 15/87

(52) U.S. Cl. 435/456; 435/459; 514/44

(57) ABSTRACT

The invention relates to the use of the gene transfer system Sleeping Beauty for the somatic gene transfer for the purpose of stably inserting DNA in the chromosomes of living vertebrates, comprising the two components of the transfer system Sleeping Beauty that are injected into the somatic cells of an animal for the purpose of gene therapy.

Considerable effort has been devoted to the development of in vivo gene delivery strategies for the treatment of inherited and aquired disorders in humans (somatic gene transfer) as well as for transgenesis of certain vertebrate species for agricultural and medical biotechnology (germline gene transfer). For effective gene therapy it is necessary to: 1) achieve delivery of therapeutic genes at high efficiency specifically to relevant cells, 2) express the gene for a prolonged period of time, 3) ensure that the introduction of the therapeutic gene is not deleterious.

There are several methods and vectors in use for gene delivery for the purpose of human gene therapy (Verma and Somia,1997). These methods can be broadly classified as viral and nonviral technologies, and all have advantages and limitations; none of them providing a perfect solution. In general, vectors that are able to integrate the transgene have the capacity to provide prolonged expression as well. On the other side, random integration into chromosomes is a concern, because of the potential disruption of endogenous gene function at and near the insertion site.

Adapting viruses for gene transfer is a popular approach, but genetic design of the vector is restricted due to the constraints of the virus in terms of size, structure and regulation of expression. Retroviral vectors (Miller, 1997) are efficient at integrating foreign DNA into the chromosomes of transduced cells, and have enormous potential for life-long gene expression. However, the amount of time and financial resources required for their preparation may not be amenable to industrial-scale manufacture. Furthermore, there are several other considerations including safety, random chromosomal integration and the requirement of cell replication for integration. Lentiviral systems, based on the human immunodeficiency virus (HIV) belong to retroviruses, but they can infect both dividing and non-dividing cells. Adenovirus vectors have been shown to be capable of in vivo gene delivery of transgenes to a wide variety of both dividing and non-dividing cells, as well as mediating high level, but short term transgene expression. Adenoviruses lack the ability to integrate the transferred gene into chromosomal DNA, and their presence in cells is short-lived. Thus, recombinant adenovirus vectors have to be administered repeatedly, generating an undesirable immune response in humans, due to the immunogenity of the vector. Adeno Associated Virus (AAV) vectors have several potential advantages to be explored, including the potential of targeted integration of the transgene. One of the obvious limitations of the AAV vehicle is the low maximal insert size (3.5-4.0 kb). Currently, combination (hybrid) vectors (retroviral/adenoviral, retroviral/AAV, etc.) have been developed that are able to address certain problems of the individual viral vector systems.

Nonviral methods, including DNA condensing agents, liposomes, microinjection and “gene guns” might be easier and safer to use than viruses. However, the efficiency of naked DNA entry and uptake is low, that can be increased by using liposomes. In general, the currently used non-viral systems are not equipped to promote integration into chromosomes. As a result, stable gene transfer frequencies using nonviral systems have been very low. Moreover, most nonviral methods often result in concatamerization as well as random breaks in input DNA, which might lead to gene silencing.

### PROBLEM TO BE ADDRESSED

Currently, there is no gene delivery system in vertebrates for somatic and germline gene transfer which would combine the following characteristics: 1) ability to transfer genes in vivo; 2) wide host- and tissue-range; 3) stable insertion of genes into chromosomes; 3) faithful, long-term expression of transferred genes; 4) safety; 5) cost-effective large-scale manufacture.

### DESCRIPTION

Transposable elements, or transposons in short, are mobile segments of DNA that can move from one locus to another within genomes (Plasterk et al., 1999). These elements move via a conservative, “cut-and-paste” mechanism: the transposase catalyzes the excision of the transposon from its original location and promotes its reintegration elsewhere in the genome. Transposase-deficient elements can be mobilized if the transposase is provided in trans by another transposase gene. Thus, transposons can be harnessed as vehicles for bringing new phenotypes into genomes by transgenesis. They are not infectious and due to the necessity of adaptation to their host, they thought to be less harmful to the host than viruses.

DNA transposons are routinely used for insertional mutagenesis, gene mapping, and gene transfer in well-established, non-vertebrate model systems such as Drosophila melanogaster or Caenorhabditis elegans, and in plants. However, transposable elements have not been used for the investigation of vertebrate genomes for two reasons. First, until now, there have not been any well-defined, DNA-based mobile elements in these species. Second, in animals, a major obstacle to the transfer of an active transposon system from one species to another has been that of species-specificity of transposition due to the requirement for factors produced by the natural host.

Sleeping Beauty (SB) is an active Tc1-like transposon that was reconstructed from bits and pieces of inactive elements found in the genomes of teleost fish. (SB) is currently the only active DNA-based transposon system of vertebrate origin that can be manipulated in the laboratory using standard molecular biology techniques. SB mediates efficient and precise cut-and-paste transposition in fish, frog, and many mammalian species including mouse and human cells (Ivics et al., 1997; Luo et al., 1998; Izsvak et al., 2000; Yant et al., 2000).

Some of the main characteristics of a desirable transposon vector are: ease of use, relatively wide host range, little size or sequence limitations, efficient chromosomal integration, and stable maintenance of faithful transgene expression throughout multiple generations of transgenic cells and organisms. Sleeping Beauty fulfills these requirements based on the following findings.

Experimental Results

Sleeping Beauty is active in diverse vertebrate species. To assess the limitations of host specificity of SB among vertebrates, cultured cells of representatives of different vertebrate classes were subjected to our standard transposition assay. Cell lines from seven different fish species, three from mouse, two from human and one each from a frog, a quail, a sheep, a cow, a dog, a rabbit, a hamster and a monkey were tested. As summarized in Table 1, SB was able to increase the frequency of transgene integration in all of these cell lines, with the exception of the quail. Thus, we concluded that SB would be active in essentially any vertebrate species (Izsvak et al., 2000).

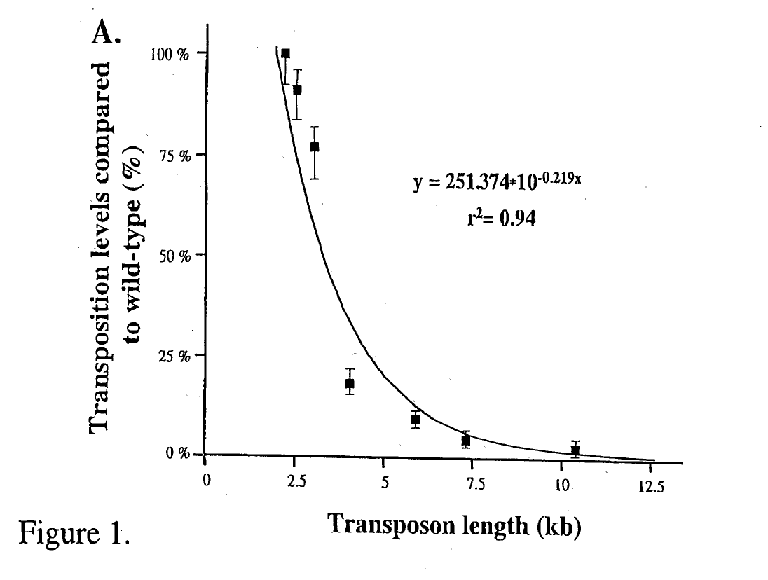

Effects of transposon size on the efficiency of Sleeping Beauty transposition. The natural size of SB is about 1.6 kb. To be useful as a vector for somatic and germline transformation, a transposon vector must be able to incorporate large (several kb) DNA fragments containing complete genes, and still retain the ability to be efficiently mobilized by a transposase. In order to determine the size-limitations of the SB system, a series of donor constructs containing transposons of increasing length (2.2; 2.5; 3.0; 4.0; 5.8; 7.3 and 10.3 kb) was tested. Similarly to other transposon systems, larger elements transposed less efficiently, and with each kb increase in transposon length we found an exponential decrease of approximately 30% in efficiency of transposition (FIG. 1) (Izsvak et al., 2000). The maximum size of SB vectors, similarly to most retroviral vectors, was found to be about 10 kb. However, although efficiency of transposition appears to decrease with increasing vector size as a general rule, the upper limit does not appear to be as strict as for retroviral vectors. Moreover, a decrease of length of DNA outside the transposon increases the efficiency of transposition as a general rule (˜30% increase/kb) (Izsvak et al., 2000). In other words, at a given insert size the transposition efficiency can be increased by bringing the two inverted repeats of the transposon closer on a circular plasmid molecule.

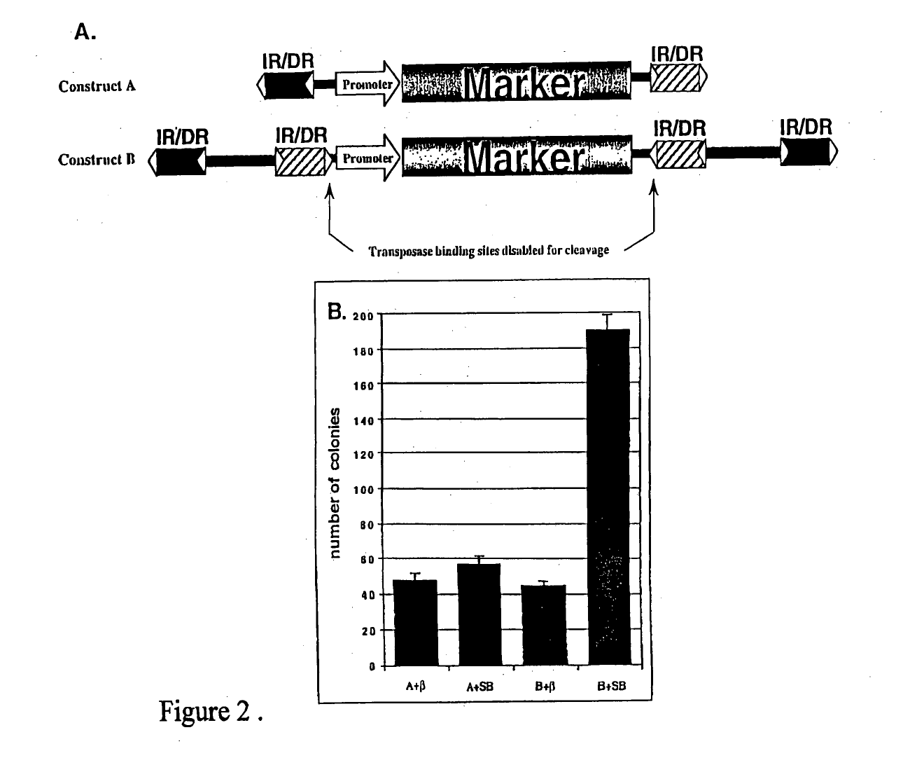

A 14 kb piece of DNA, flanked by a pair of Paris elements, appears to have transposed in Drosophila virilis. We hypothesized that this kind of “sandwich” arrangements of two complete SB elements flanking a transgene will increase the ability of the vector to transpose larger pieces of DNA. Thus, we flanked an approximately 5 kb piece of DNA with two intact copies of SB in an inverted orientation (FIG. 2A). The vector was designed in a way that transposase was able to bind to its internal binding sites within each element but its ability to cleave DNA at those sites was abolished. Efficiency of transposition of the sandwich element was about 4-fold increased compared to an SB vector containing the same marker gene (FIG. 2B) (unpublished results). Thus, the sandwich transposon vector can be useful to extend the cloning capacity of SB elements for the transfer of large genes whose stable integration into genomes has been problematic with current viral and nonviral vectors.

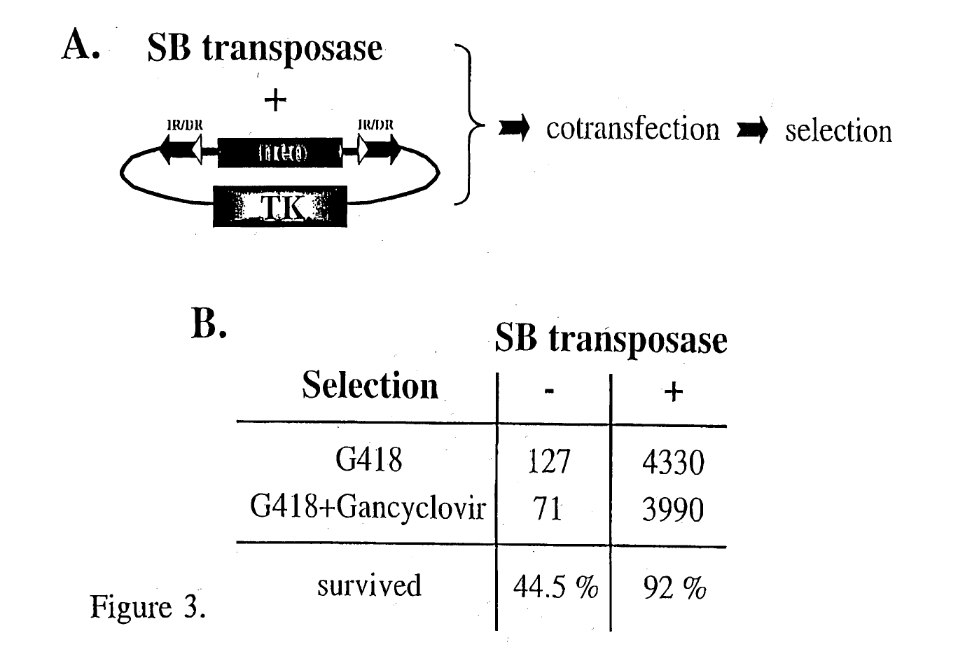

Sleeping Beauty integrates in a precise manner. Our analysis of a handful, randomly chosen SB insertion sites in HeLa cells revealed that chromosomal integration was precise in all of the cases, and was accompanied by duplication of TA target dinucleotides (Ivics et al., 1997), a molecular signature of Tc1/mariner transposition. To determine the ratio of precise versus non-precise integration events in a lareger scale, a genetic assay for positive-negative selection was devised. This assay positively selects for integration of transposon sequences (precise events), and negatively selects against cells that carry integrated vector sequences in their chromosomes (non-precise events). The thymidine kinase (TK) gene of herpes simplex virus type 1 was built into the vector backbone of pT/neo. Upon cotransfection of this construct into cells together with a SB transposase-expressing plasmid, G-418-resistant colonies are selected either in the presence or absence of gancyclovir, which is toxic to cells expressing the TK gene. About 90% of the G-418-resistant Hela colonies survived gancyclovir selection, indicating that the majority of the integration events did not include the toxic TK gene, which is a measure of precise, transposase-mediated integration events (FIG. 3). Similar results, indicative of precise transposition, were obtained in hamster K1, fathead minnow FHM and mouse 3T3 cells (Izsvak et al., 2000). Our results indicate a high fidelity of substrate recognition and precise transposition of SB even in these phylogenetically distant cell lines. The SB system provides precise integration of the desired gene, flanked by the short inverted repeat sequences (230 bp) only. This fidelity of integration means that plasmid sequences carrying antibiotic resistance genes are left behind and are not integrating into the host genome, addressing a general problem concerning gene therapy and transgenesis.

In contrast to concatamerization of extrachromosomal DNA, which is often encountered using nonviral gene transfer methods, SB transposons integrate as single copies.

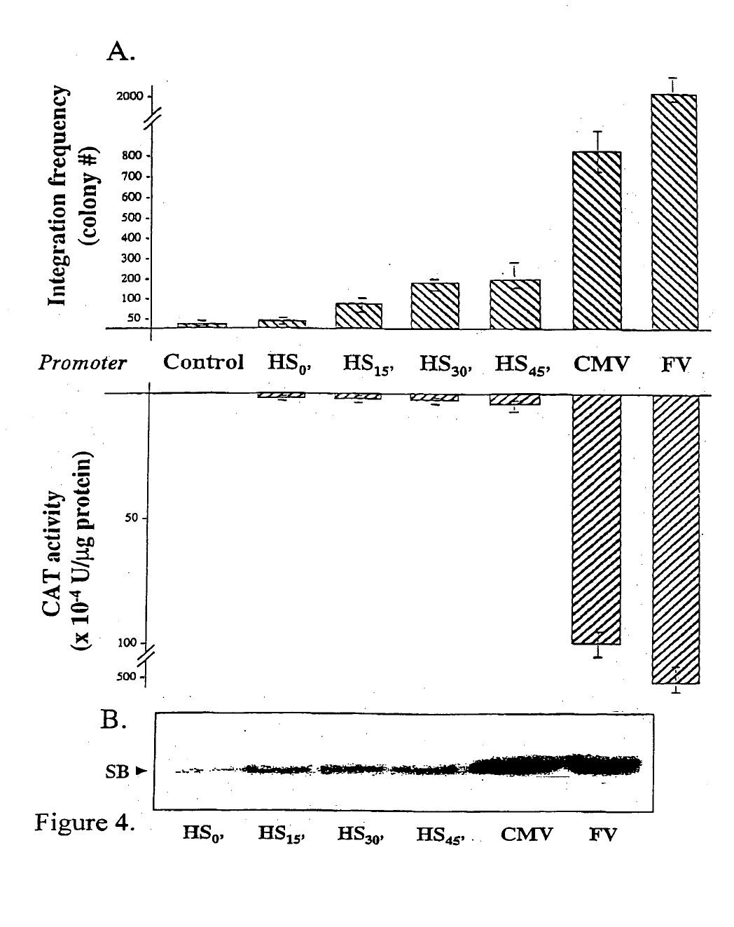

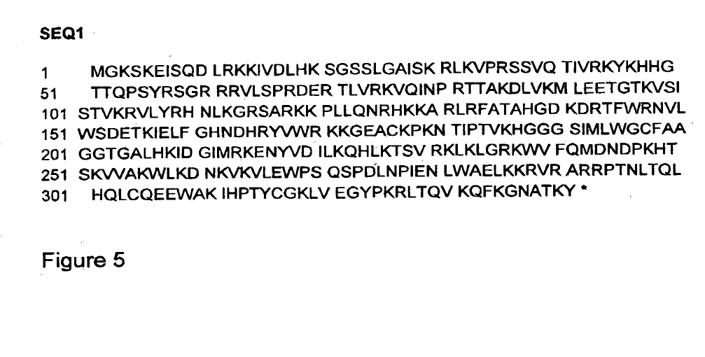

SB can be expressed from a wide range of promoters to optimize transposase expression for a variety of applications. Three different promoters were used to express SB transposase, those of the human heat shock 70 (HS) gene, the human cytomegalovirus (CMV) immediate early gene and the carp β-actin gene (FV). HS is inducible by applying heat shock on transfected cells, whereas CMV and FV can be considered as 'strong” constitutive promoter. As shown in the upper graph of FIG. 4, using HS-SB and by increasing the time of the induction (15 min, 30 min and 45 min), the numbers of G-418-resistant colonies increased as well. The CMV promoter-driven transposase produced a significantly higher number of colonies, and we obtained even higher numbers with FV-SB (Izsvak et al., 2000). We assessed the relative strengths of the three promoters in gene expression by measuring chloramphenicol acetyl transferase (CAT) reporter enzyme activity from transiently transfected cells. Levels of CAT activity, when expressed from the same promoters under the same experimental conditions, showed about the same ratios as those we obtained for transpositional activities (FIG. 4, lower graph).

We concluded that the number of transposition events per transfected cell population is directly proportional to the number of transposase molecules present in cells. Thus, overexpression of transposase does not appear to have an inhibitory effect on SB transposition, at least not in the range of expression in which SB would be used in most transgenic experiments, and thus SB can be expressed from a wide range of promoters to optimize transposase expression for a variety of applications.

Sleeping Beauty transposon mediates the insertion of foreign genes into the genomes of vertebrates in vivo. In contrast to viral vectors, tremendous quantities of plasmid-based vectors can be readily produced, purified and maintained at very little cost. Sleeping Beauty is is the first non-viral system that allows plasmid-encoded gene integration and long-term expression in vivo.

Using naked DNA, tail-vein injection technique, Sleeping Beauty transposase was shown to efficiently mediate transposon integration into multiple non-coding regions of the mouse genome in vivo. DNA transposition occurs in approximately 5-6 percent of transfected mouse cells and results in long term expression (>3 month) of therapeutic levels of human clotting factor IX in vivo (Yant et al., 2000). These results establish DNA-mediated transposition as a powerful new genetic tool for vertebrates and provide intriguing new stategies to improve existing non-viral and viral vectors for transgenesis and for human gene therapy applications.

The Sleeping Beauty inverted repeat sequences do not carry promoter and/or enhancer elements, which can potentially influence neighbouring gene expression upon integration into the genome. To test whether the inverted repeat sequence of the Sleeping Beauty transposon carries promoter elements, the following experiment was performed. The lacZ gene was fused in frame to the SB transposase gene in a construct that retained the transposon inverted repeat sequences upstream the expression unit. Human HeLa cells transfected with this construct were either stained in situ or cell extracts were tested for β-galactosidase activity in an in vitro assay. No detectable β-galactosidase activity was obtained in either case, suggesting that no significant promoter activity could be rendered to the inverted repeats.

To test for enhancer activity, the left inverted repeat of the SB transposon was fused to a minimal TK promoter in front of the luciferase marker gene. The human cytomegalovirus (CMV) enhancer served as a positive control. No significant enhancer activity was observed from the inverted repeat sequence of Sleeping Beauty (unpublished results). Thus, in contrast to retroviruses whose LTRs contain enhancer/promoter elements, SB vectors are transcriptionally neutral, and thus would not alter patterns of endogenous gene expression.

Single amino acid replacements at nonessential positions in the transposase polypeptide do not alter transposase activity. Eukaryotic expression plasmids are all derivatives of the pCMV/SB construct described earlier (Ivics et al., 1997). pCMV/SB-S116V was made by PCR-amplification of pCMV/SB with primers

5′-CCGCGTCGCGAGGAAGAAGCCACTGCTCCAA-3′
and
5′-CTTCCTCGCGACGCGGCCTTTCAGGTTATGTCG-3′,

cutting the PCR product with restriction enzyme NruI whose recogition sequence is underlined within the primer sequences, and recircularization with T4 DNA ligase. The mutant sequence with the encoded amino acids is the following:

109 110 111 112 113 114 115 116 117 118 119 120 121 122 123 124
CGA CAT AAC CTG AAA GGC CGC GTC GCG AGG AAG AAG CCA CTG CTC CAA
R H N L K G R V A R K K P L L Q

The mutation is a single amino acid change in position 116, which is now a valine (typed bold) in place of the original serine.

pCMV/SB-N280H was made by PCR-amplification of pCMV/SB with primers

5′-GCCCAGATCTCAATCCTATAGAACATTTGTGGGCAGAACTG-3′
and
5′-ATTGAGATCTGGGCTTTGTGATGGCCACTCC-3′,

cutting the PCR product with restriction enzyme BglII whose recogition sequence is underlined within the primer sequences, and recircularization with T4 DNA ligase. Part of he mutant sequence with the encoded amino acids is the following:

270 271 272 273 274 275 276 277 278 279 280 281 282 283 284 285
TCA CAA AGC CCA GAT CTC AAT CCT ATA GAA CAT TTG TGG GCA GAA CTG
S Q S P D L N P I E H L W A E L

The mutation is a single amino acid change in position 280, which is now a histidine (typed bold) in place of the original asparagine.

pCMV/SB-S58P was made by PCR amplification of a DNA fragment across the junction of the CMV promoter and the transposase gene in pCMV/SB with primers

5′-GGTGGTGCAAATCAAAGAACTGCTCC-3′
and
5′-CAGAACGCGTCTCCTTCCTGGGCGGTATGACGGC-3′,

digestion with EagI which cuts at the junction of the CMV promoter and the transposase gene and MluI (underlined), and cloning into the respective sites in pCMV/SB. Part of he mutant sequence with the encoded amino acids is the following:

```
  54 55 56 57 58 59 60 61 62
G CCG TCA TAC CGC CCA GGA AGG AGA CGC GT
  P S Y R P G R R R
```

The mutation is a single amino acid change in position 58, which is now a proline (typed bold) in place of the original serine.

All constructs carrying the mutations were checked for proper expression by Western hybridizations, using an anti-SB polyclonal antibody. All three above mutant transposases mediate transposition using the pT/neo donor construct (Ivics et al., 1997) in human Hela cells at comparable levels to wild-type SB transposase (unpublished results). Comparable level is defined here being within a range of 90% to 110% of the activity of wild-type SB transposase. Alltogether these data demonstrate that directed changes can be introduced into the transposase polypeptide without negatively affecting its functional properties. In summary, the SB system has several advantages for gene transfer in vertebrates:
SB can transform a wide range of vertebrate cells;
because SB is a DNA-based transposon, there is no need for reverse transcription of the transgene, which introduces mutations in retroviral vector stocks;
SB does not appear to be restricted in its ability to transpose DNA of any sequence;
SB vectors do not have strict size limitations;
since transposons are not infectious, transposon-based vectors are not replication-competent, herefore do not spread to other cell populations;
SB requires only about 230 bp transposon inverted repeat DNA flanking a transgene on each side for mobilization;
SB vectors are transcriptionally neutral, and thus do not alter endogenous gene function;
transposition is inducible, and requires only the transposase protein, thus one can simply control the site and moment of jumping by control of transposase expression.
SB is expected to be able to transduce nondividing cells, because the transposase contains a nuclear localization signal, through which transposon/transposase complexes could be actively transported into cell nuclei;
SB mediates stable, single-copy integration of genes into chromosomes which forms the basis of long-term expression throughout multiple generations of transgenic cells and organisms;
once integrated, SB elements are expected to behave as stable, dominant genetic determinants in the genomes of transformed cells, because 1) the presence of SB transposase is only transitory in cells and is limited to a time window when transposition is catalyzed, and 2) there is no evidence of an endogenous transposase source in vertebrate cells that could activate and mobilize integrated SB elements;
with the exception of some fish species, there are no endogenous sequences in vertebrate genomes with sufficient homology to SB that would allow recombination and release of transpositionally competent (autonomous) elements;
for efficient introduction into cells, SB could be combined with DNA delivery agents such as adenoviruses and liposomes;

because SB is a plasmid-based vector, its production is easy, inexpensive, and can be scaled up.

TABLE 1

| | | | Transposase | | |
|---|---|---|---|---|---|
| Class | Organism | Cell line | − | + | Activity |
| Mammals | Human | Hela | 282 | 8750 | +++++ |
| | | Jurkat | 2 | 6 | + |
| | Monkey | Cos-7 | 885 | 1845 | + |
| | Mouse | LMTK | 155 | 805 | ++ |
| | | 3T3 | 170 | 850 | ++ |
| | | ES (AB1)^{#} | | | ++ |
| | Hamster | K1 | 8250 | 87900 | ++++ |
| | Rabbit | SIRC | 174 | 318 | + |
| | Dog | MDCK-II | 22 | 50 | + |
| | Cow | MDBK | 480 | 4185 | +++ |
| | Sheep | MDOK | 13 | 27 | + |
| Birds | Quail | QT6 | 4 | 3 | ? |
| Amphibians | Xenopus | A6 | 12 | 252 | +++++ |
| Fishes | Zebrafish | ZF4 | 7 | 13 | + |
| | Carp | EPC | 54 | 129 | + |
| | Sea bream | SAF1 | 9 | 13 | + |
| | Medaka | OLF136 | 10 | 34 | ++ |
| | Trout | RTG | 4 | 13 | + |
| | Swordtail | A2 | 37 | 108 | + |
| | Fathead minnow | FHM | 4 | 104 | +++++ |

1. A method for using the Sleeping Beauty gene transfer system for somatic gene transfer for stable insertion of DNA into chromosomes of living vertebrates comprising:
the two components of the Sleeping Beauty transfer system which are transferred into the somatic cells of an animal for gene therapeutic purposes.

2. Wherein the method of claim 1 is used for diagnostic purposes.

3. The method of claim 1 wherein the transferable gene inserted into the Sleeping Beauty transposon is from human.

4. The method of claim 1 wherein the transferable gene inserted into the Sleeping Beauty transposon is from fish.

5. The method of claim 1 wherein the transferable gene inserted into the Sleeping Beauty transposon is from frog.

6. The method of claim 1 wherein the transferable gene inserted into the Sleeping Beauty transposon is from reptile.

7. The method of claim 1 wherein the transferable gene inserted into the Sleeping Beauty transposon is from bird.

8. The method of claim 1 wherein the transferable gene inserted into the Sleeping Beauty transposon is from mammals.

9. The method of claim 1 wherein the vertebrate is a human.

10. The method of claim 1 wherein the vertebrate is a fish.

11. The method of claim 1 wherein the vertebrate is a frog.

12. The method of claim 1 wherein the vertebrate is a reptile.

13. The method of claim 1 wherein the vertebrate is a bird.

14. The method of claim 1 wherein the vertebrate is a mammal.

15. The method of claim 1 wherein the transposase source is a transposase protein.

16. The method of claim 1 wherein the transposase source is an mRNA.

17. The method of claim 1 wherein the transposase source is a plasmid DNA.

18. The method of claim 1 wherein the delivery method is a gene gun.

19. The method of claim 1 wherein the Sleeping Beauty transfer system is combined with liposomes.

20. The method of claim 1 wherein the Sleeping Beauty transfer system is combined with PEI (polyethylene Imine).

21. The method of claim 1 wherein the Sleeping Beauty transfer system is combined with adenovirus-polylysine-DNA complexes-receptor-mediated gene transfer.

22. The method of claim 1 wherein the Sleeping Beauty transfer system is combined with a recombinant retrovirus.

23. The method of claim 1 wherein the delivery method is transduction.

24. The method of claim 1 wherein the Sleeping Beauty transfer system is combined with a recombinant adenovirus.

25. The method of claim 1 wherein the delivery method is infection.

26. The method of claim 1 wherein the Sleeping Beauty transfer system is combined with a recombining herpes virus.

27. The method of claim 1 wherein the Sleeping Beauty transfer system is combined with a recombinant adeno-associated virus.

28. The method of claim 1 characterised in that the transferable gene is flanked by two complete SB elements in either direct or inverted orientation with respect to each other, characterised in that an SB element is defined by two IR/DR sequences in an inverted orientation with respect to each other, each containing at least two transposase binding sites.

29. The method of claim 1 characterised in that the transposase has at least a 90%, preferably a 95%, more preferably a 98% sequence identity to SEQ1.

30. The method or claim 1 wherein the transferable gene is used for correcting a single gene defect.

31. The method of claim 1 wherein the therapeutic gene is used in cancer gene therapy.

32. The method for using the Sleeping Beauty transfer system for germ line gene transfer in vertebrate organisms comprising:
a. the two components of the Sleeping Beauty transfer system which are introduced into the germ stem cell to yield a transgenic cell.
b. growing the transgenic cell into a transgenic animal.

33. The method of claim 32 wherein the cell is plurlpotent or totipotent.

34. The method of claim 32 wherein delivery method is microinjection.

35. The method of claim 32 wherein the delivery method is gene gun.

36. The method of claim 32 wherein the delivery method is germ transport of the Sleeping Beauty transfer system.

37. The method of claim 32 characterised in that the transferable gene is flanked by two complete SB elements in either direct or inverted orientation with respect to each other, characterised in that an SB element; is defined by two IR/DR sequences in an inverted orientation with respect to each other, each containing at least two transposase binding sites.

38. The method of claim 32 characterised in that the transposase has at least a 90%, preferably a 95%, more preferably a 98% sequence identity to SEQ1.

39. The method of claim 32 wherein the transferable gene inserted into the Sleeping Beauty transposon is from human.

40. The method of claim 32 wherein the transferable gene inserted into the Sleeping Beauty transposon is from fish.

41. The method of claim 32 wherein the transferable gene inserted into the Sleeping Beauty transposon is from frog.

42. The method of claim 32 wherein the transferable gene inserted into the Sleeping Beauty transposon is from reptile.

43. The method of claim 32 wherein the transferable gene inserted into the Sleeping Beauty transposon is from bird.

44. The method of claim 32 wherein the transferable gene inserted into the Sleeping Beauty transposon is from mammals.

45. The method of claim 32 wherein the vertebrate is a human.

46. The method of claim 32 wherein the vertebrate is a fish.

47. The method of claim 32 wherein the vertebrate is a frog.

48. The method of claim 32 wherein the vertebrate is a reptile.

49. The method of claim 32 wherein the vertebrate is a bird.

50. The method of claim 32 wherein the vertebrate is a mammal.

51. The method of claim 32 wherein the transposase source is a transposase protein.

52. The method of claim 32 wherein the transposase source is an mRNA.

53. The method of claim 32 wherein the transposase source is a plasmid DNA.
